SEQUENCE LISTING (19 sequences)

SEQ ID NO: 1 - protein, 510 aa, unidentified
  source 1..510: note = human Nectin-4
  REGION 1..510: note = Nectin-4
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 2 - protein, 118 aa, synthetic construct
  REGION 1..118: note = variable heavy chain
QVQLVQSGAEVKKPGASVKVSCKASGFTFNSMYIHWVRQAPGQRLEWMGWIYAGTGGTRYSQKFQGRVTITRDTSASTAYMELSSLRSEDTAVYYCAIRSGFVPMDYWGQGTLVTVSS

SEQ ID NO: 3 - protein, 118 aa, synthetic construct
  REGION 1..118: note = variable heavy chain
QVQLVQSGAEVKKPGASVKVSCKASGFTFNSMYISWVRQAPGQRLEWMAWIYAGTGGTRYSQKFQGRVTITRDTSASTAYMELSSLRSEDTAVYYCAIRSGFVPMDYWGQGTLVTVSS

SEQ ID NO: 4 - protein, 118 aa, synthetic construct
  REGION 1..118: note = variable heavy chain
QVQLVQSGAEVKKPGASVKVSCKASGFTFNSMYISWVRQAPGQRLEWMAWIYAGTGGTRFNQKFTGKVTITRDTSASTAYMELSSLRSEDTAVYYCAIRSGFVPMDYWGQGTLVTVSS

SEQ ID NO: 5 - protein, 118 aa, synthetic construct
  REGION 1..118: note = variable heavy chain
QVQLVQSGAEVKKPGASVKVSCKASGFTFNSMYISWVRQAPGQSLEWMAWIYAGTGGTRFNQKFTGKVTITVDTSASTAYMELSSLRSEDTAVYYCAIRSGFVPMDYWGQGTLVTVSS

SEQ ID NO: 6 - protein, 107 aa, synthetic construct
  REGION 1..107: note = variable light chain
DIVMTQSPDSLAVSLGERATINCKSSQSVSNDVAWYQQKPGQPPKLLIYYASNRESGVPDRFSGSGSGTDFTLTISSLQAEDVAVYYCQQDYSSPWTFGQGTKLEIK

SEQ ID NO: 7 - protein, 107 aa, synthetic construct
  REGION 1..107: note = variable light chain
DIVMTQSPDSLAVSLGERATINCKASQSVSNDVAWYQQKPGQPPKLLIYYASNRESGVPDRFSGSGSGTDFTLTISSLQAEDVAVYYCQQDYSSPWTFGQGTKLEIK

SEQ ID NO: 8 - protein, 107 aa, synthetic construct
  REGION 1..107: note = variable light chain
DIVMTQSPDSLAVSLGERATINCKASQSVSNDVAWYQQKPGQPPKLLIYYASNRYTGVPDRFSGSGSGTDFTLTISSLQAEDVAVYYCQQDYSSPWTFGQGTKLEIK

SEQ ID NO: 9 - protein, 107 aa, synthetic construct
  REGION 1..107: note = variable light chain
DIVMTQSPDSLAVSLGERATINCKASQSVSNDVAWYQQKPGQSPKLLIYYASNRYTGVPDRFSGSGSGTDFTLTISSLQAEDVAVYYCQQDYSSPWTFGQGTKLEIK

SEQ ID NO: 10 - protein, 5 aa, synthetic construct
  REGION 1..5: note = complementarity-determining-region-H1
SMYIH

SEQ ID NO: 11 - protein, 5 aa, synthetic construct
  REGION 1..5: note = complementarity-determining-region-H1
SMYIS

SEQ ID NO: 12 - protein, 17 aa, synthetic construct
  REGION 1..17: note = complementarity-determining-region-H2
WIYAGTGGTRYSQKFQG

SEQ ID NO: 13 - protein, 17 aa, synthetic construct
  REGION 1..17: note = complementarity-determining-region-H2
WIYAGTGGTRFNQKFTG

SEQ ID NO: 14 - protein, 9 aa, synthetic construct
  REGION 1..9: note = complementarity-determining-region-H3
RSGFVPMDY

SEQ ID NO: 15 - protein, 11 aa, synthetic construct
  REGION 1..11: note = complementarity-determining-region-L1
KSSQSVSNDVA

SEQ ID NO: 16 - protein, 11 aa, synthetic construct
  REGION 1..11: note = complementarity-determining-region-L1
KASQSVSNDVA

SEQ ID NO: 17 - protein, 7 aa, synthetic construct
  REGION 1..7: note = complementarity-determining-region-L2
YASNRES

SEQ ID NO: 18 - protein, 7 aa, synthetic construct
  REGION 1..7: note = complementarity-determining-region-L2
YASNRYT

SEQ ID NO: 19 - protein, 9 aa, synthetic construct
  REGION 1..9: note = complementarity-determining-region-L3
QQDYSSPWT

(19) United States
(12) Patent Application Publication
(10) Pub. No.: US 2026/0317410 A1
(43) Pub. Date: Oct. 15, 2026

(54) HUMANIZED ANTI-NECTIN-4 ANTIBODIES

(71) Applicants: ELI LILLY AND COMPANY, Indianapolis, IN (US); UNIVERSITÉ D'AIX-MARSEILLE, Marseille (FR); INSERM (Institut National De La Sante Et De La Recherche Medicale), Paris (FR); CENTRE NATIONAL DE LA RECHERCHE SCIENTIFIQUE - CNRS, Paris (FR); INSTITUT JEAN PAOLI & IRENE CALMETTES, Marseille (FR)

(72) Inventors: Jack ELANDS, Brussels (BE); Florence LHOSPICE, Primelin (FR); Marc LOPEZ, Marseille (FR); Daniel OLIVE, Marseille (FR); Xavier PRÉVILLE, Roquefort les Pins (FR)

(21) Appl. No.: 19/104,324

(22) PCT Filed: Aug. 16, 2023
(86) PCT No.: PCT/EP2023/072528

§ 371 Date: Feb. 17, 2025

(30) Foreign Application Priority Data
Aug. 18, 2022 (EP) 22191026.8

(51) Int. Cl. C07K 16/18; A61K 49/00; A61K 51/10

(52) U.S. Cl. CPC C07K 16/18; A61K 49/0002; A61K 51/1018; A61K 2123/00; C07K 2317/24

(57) ABSTRACT

The present invention relates to humanized antibodies having specificity to Nectin-4 and uses thereof.

### BACKGROUND INFORMATION

Nectins are adhesion molecules that help organize epithelial and endothelial junctions and serve as receptors for the entry of the herpes simplex virus, measle virus and poliovirus.

Nectins belong to the immunoglobulin superfamily and are homologs of the poliovirus receptor (PVR/CD155), and for this, have also been called poliovirus receptor bound proteins (PRR). To date, 5 members have been described PVR/CD155, Nectin-1/PRR1/CD111, Nectin-2/PRR2/CD112, Nectin-3/PRR3 and Nectin-4/PRR4. Their ectodomain is made up of three immunoglobulin-like (Ig)-type V, C, C domains that share between 30 and 55% identity in their amino acid sequences.

Expression of Nectin/PRR molecules is generally extensive in tissues, including hematopoietic, neuronal, endothelial and epithelial cells, with the exception of Nectin-3 and -4, which exhibit more restricted expression profiles.

Nectin-4 is a particularly interesting target. It is expressed during fetal development, but its expression decreases and is very restricted in adult tissues in comparison to that of other members of the Nectin family. Nectin-4 is a tumor associated antigen in 83% of bladder cancers, 78% of breast cancers (mainly triple negative and ERBB2+), 71% Pancreatic cancers, 55% of lung cancers, 57% of ovarian cancers, 59% of head and neck cancers, and 55% of esophageal cancers.

Expression of Nectin-4 in these pathologies is associated with poor prognosis, probably because of the ability of Nectin-4 to confer to tumor cells in vitro, higher capacities of migration, proliferation and formation of metastases.

In normal tissues, Nectin-4 is only detected in skin, salivary glands, urinary bladder and esophagus. The recent approval by heath authorities of Enfortumab vedotin for the 2^{nd} line treatment of advanced urothelial cancer has completed the validation of Nectin-4 as a target for the treatment of cancer.

### DETAILED DESCRIPTION OF THE INVENTION

The present invention relates to humanized antibodies having specificity to Nectin-4, antigen-binding fragments thereof as well as uses thereof.

In particular, the present invention provides humanized antibodies that derive from the monoclonal anti-Nectin-4 antibody 5A12.2 mAb.

The humanized variants derived from 5A12.2 which can be provided with high yields represent a new way to improve therapeutic index of Nectin-4 positive cancer treatment combined with reduced skin toxicity and high anti-tumor selectivity and efficacy.

Surprisingly, the antibodies of the invention can be provided with significantly higher yield compared to non-humanized antibodies. This could be shown for small as well as larger production approaches. Such reproducible production in high yields greatly facilitates pharmaceutical use.

A first aspect of the invention relates to a monoclonal antibody or an antigen-binding fragment thereof which binds to a discontinuous epitope on Nectin-4 consisting of amino acids L81, H83, Y86, G87, H89, S91, P92 and E95 of SEQ ID NO: 1 as determined using Deep Mutational Scanning (DMS). DMS is known to the person skilled in the art. Basically, every possible amino acid change in a given protein is first synthesized. The activity of each of these protein variants is assayed in parallel using barcodes for each variant. By comparing the activity to the wild-type protein, the effect of each mutation is identified. One detailed example of DMS is given herein below.

Preferably, the inventive antibodies has a better avidity for their dimeric epitope on Nectin-4 than the prior art antibody HA22. HA22 is a human monoclonal anti-Nectin-4 antibody described in WO2012/047724, the content of which is herein incorporated by reference. “Avidity” as herein described describes the measure of overall or accumulated strength of a protein-protein complex, i.e. the total strength of all the non-covalent interactions between an antibody binding to its ligand/s. It is determined by three parameters: the binding affinity of the complex, the valency of the proteins and the structural arrangement of the proteins in the complex. The inventive antibody is not the antibody mAb 5A12.2 as obtainable by the hybridoma deposited at the CNCM under the deposit number CNCM I-5407 as for example described in WO2021/069508.

The antibodies of the invention are monoclonal antibodies (mAb) or monoclonal antibody fragments characterized by a specific amino acid sequence. If not indicated differently, the term “monoclonal” refers to a single species, i.e. single amino acid composition of antibodies or antibody fragments.

The antigen-binding site of an inventive antibody comprises heavy chain variable domains/regions (VH) and/or antibody light chain variable domains/regions (VL), or pairs of VH/VL. The variable domains/region denote each of the pair of light and heavy chains, which is involved directly in binding the antibody to the antigen. The variable domain of a heavy chain is abbreviated as “VH” and the variable domain of a light chain is abbreviated as “VL”.

The term “antigen-binding site” denotes the region(s) of an antibody molecule to which a ligand (e.g. the antigen, i.e. Nectin-4, or antigen fragment of it) actually binds and which is derived from an antibody.

An antigen-binding site of an antibody according to the invention can contain six complementarity determining regions (CDRs) which contribute in varying degrees to the affinity of the binding site for the antigen. There are three heavy chain variable domain CDRs (CDR-H1, CDR-H2 and CDR-H3) and three light chain variable domain CDRs (CDR-L1, CDR-L2 and CDR-L3). Also included within the scope of the invention are functional antigen binding sites comprised of fewer CDRs (i.e., where binding specificity is determined by three, four or five CDRs). For example, less than a complete set of 6 CDRs may be sufficient for binding. In some cases, a VH or a VL domain will be sufficient.

According to the present invention, a VH region or the CDRs thereof alone may constitute a complete antigen-binding site. In certain embodiments, the antibody comprises a VH region or the CDRs thereof as defined herein alone. In other embodiments, the antibody comprises a VH region or the CDRs thereof as defined herein together with a VL region or the CDRs thereof, particularly with a VL region or the CDRs thereof as defined herein.

The position of CDRs within a VH or VL region may be defined according to Kabat, et al., Sequences of Proteins of Immunological Interest, 5th Ed. Public Health Service, National Institutes of Health, Bethesda, MD (1991) or the IMGT numbering system, both of which are known to the person skilled in the art. The IMGT numbering has been defined to compare the variable domains whatever the antigen receptor, the chain type, or the species (Lefranc M.-P., “Unique database numbering system for immunogenetic analysis” Immunology Today, 18, 509 (1997); Lefranc M.-P., “The IMGT unique numbering for Immunoglobulins, T cell receptors and Ig-like domains” The Immunologist, 7, 132-136 (1999)).

A further aspect of the present invention relates to a humanized monoclonal antibody or antigen-binding fragment thereof which binds to Nectin-4 comprising
(a) a variable heavy chain (VH) region comprising an amino acid sequence according to any one of SEQ ID NOs: 2-5, or an amino acid sequence having an identity thereto of at least 85%, at least 90%, at least 95% or at least 99%, and including complementarity-determining regions (CDRs) CDR-H1 according to SEQ ID NO: 10 or 11, CDR-H2 according to SEQ ID NO: 12 or 13 and CDR-H3 according to SEQ ID NO: 14, and
(b) a variable light chain (VL) region comprising an amino acid sequence according to any one of SEQ ID NOs: 6-9, or an amino acid sequence having an identity thereto of at least 85%, at least 90%, at least 95% or at least 99%, and including CDR-L1 according to SEQ ID NO: 15 or 16, CDR-L2 according to SEQ ID NO: 17 or 18 and CDR-L3 according to SEQ ID NO: 19.

Specific amino acid sequences represented by SEQ ID NO:s used herein are provided in the attached sequence listing. SEQ ID NO: 10-19 define the six CDR sequences of the inventive humanized antibody according to the Kabat numbering system. If not stated otherwise, the Kabat system is used herein.

According to the present invention, a substitution of 1 or 2 amino acids in these CDR sequences is possible. In particular embodiments, a conservative amino acid substitution is preferable, i.e. a substitution of an amino acid by another amino acid with similar biochemical properties, for example a substitution of an aliphatic amino acid, e.g. Gly, Ala, Val, Leu or Ile, for another aliphatic amino acid, a basic amino acid, e.g. His, Lys or Arg, against another basic amino acid, an acidic amino acid or an amide thereof, e.g. Asp, Glu, Asn or Gln, against another acidic amino acid or an amide thereof, an aromatic amino acid, e.g. Phe, Tyr or Trp, against another aromatic amino acid, or a hydroxy or sulfur containing amino acid, e.g. Ser, Thr, Met or Cys, against another hydroxy or sulfur containing amino acid.

An “antigen-binding fragment” of an antibody refers to a molecule comprising a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multi-specific antibodies formed from antibody fragments. The term also encompasses a fusion protein, e.g. a fusion protein with a non-immunoglobulin peptide or polypeptide, and a conjugate with a non-proteinaceous structure, e.g. a label or a toxin. The terms “antigen-binding fragment of an antibody”, “antigen-binding fragment thereof”, “fragment of an antibody” or “fragment thereof” may be used interchangeably herein.

The inventive antibody or the antigen-binding fragment thereof may be mono- or multivalent, i.e. it may comprise a single antigen-binding site or multiple antigen-binding sites. For example, Fab fragments have single antigen-binding site, antibodies of the IgG class or Fv or scFv fragments have two antigen-binding sites and antibodies of the IgM class have 5 antigen-binding sites. The term “antibody” also encompasses hetero-specific antibodies, e.g. hetero-bispecific antibodies, which have different antigen-binding sites, particularly antibodies, which are directed to two different epitopes on the antigen. As used herein, “epitope” is a region of an antigen that is bound by an antibody. The term “epitope” includes any polypeptide determinant capable of specific binding to an antibody.

According to another preferred embodiment, the humanized antibody or antigen-binding fragment thereof comprises
(a) a VH region comprising an amino acid sequence according to SEQ ID NO:2-5, and
(b) a VL region comprising an amino acid sequence according to SEQ ID NO: 6-9.

More particularly, the antibody or antigen-binding fragment thereof may comprise
(i) a VH region comprising an amino acid sequence according to SEQ ID NO: 2 or an amino sequence having an identity thereof of at least 85%, at least 90%, at least 95% or at least 99%, and a VL region comprising an amino acid sequence according to SEQ ID NO: 6 or an amino sequence having an identity thereof of at least 85%, at least 90%, at least 95% or at least 99%,
(ii) a VH region comprising an amino acid sequence according to SEQ ID NO: 3, or an amino sequence having an identity thereof of at least 85%, at least 90%, at least 95% or at least 99%, and a VL region comprising an amino acid sequence according to SEQ ID NO: 7 or an amino sequence having an identity thereof of at least 85%, at least 90%, at least 95% or at least 99%,
(iii) a VH region comprising an amino acid sequence according to SEQ ID NO:4 or an amino sequence having an identity thereof of at least 85%, at least 90%, at least 95% or at least 99%, and a VL region comprising an amino acid sequence according to SEQ ID NO: 8, or an amino sequence having an identity thereof of at least 85%, at least 90%, at least 95% or at least 99%,
(iv) a VH region comprising an amino acid sequence according to SEQ ID NO: 5, or an amino sequence having an identity thereof of at least 85%, at least 90%, at least 95% or at least 99%, and a VL region comprising an amino acid sequence according to SEQ ID NO: 9, or an amino sequence having an identity thereof of at least 85%, at least 90%, at least 95% or at least 99%.

“Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, Alignment for purposes of determining percent amino acid sequence identity can be achieved in various ways that are within the skill in the art, for instance, using publicly available computer software such as BLAST.

The antibody of the invention may be a chimeric antibody, a multispecific antibody, in particular a bispecific antibody, a human antibody, a humanized antibody or an antigen-binding fragment thereof.

According to the present invention, a “chimeric” antibody refers to an antibody in which a portion of the heavy and/or light chain is derived from a particular source or species, while the remainder of the heavy and/or light chain is derived from a different source or species.

“Multispecific antibodies” bind two or more different epitopes. The epitopes may be on the same or different antigens. A preferred example of a multispecific antibody is a “bispecific antibody” which binds two different epitopes

The term “humanized antibody” or “humanized version of an antibody” refers to antibodies for which both heavy and light chains are humanized as a result of antibody engineering. A humanized chain is typically a chain in which the V-region amino acid sequence has been changed so that, analyzed as a whole, is closer in homology to a human germline sequence than to the germline sequence of the species of origin. For example, a murine CDR may be grafted into the framework region of a human antibody to prepare the “humanized antibody.” See, e.g., Riechmann, L., et al., Nature 332 (1988) 323-327; and Neuberger, M. S., et al., Nature 314 (1985) 268-270. Other forms of humanized antibodies encompassed by the present invention are those in which the constant region has been additionally modified or changed from that of the original antibody to generate the properties according to the invention. Humanization assessment is based on the resulting amino acid sequence and not on the methodology per se.

The antibody of the present invention may be of any suitable class. The term “class” refers to the type of constant domain or constant region possessed by its heavy chain. As used herein, “constant domain” or “constant region” denotes the sum of the domains of an antibody other than the variable region. The constant region is not directly involved in binding of an antigen, but exhibits various effector functions. The antibody may be of any of the five major classes of antibodies: IgA, IgD, IgE, IgG, and IgM, or any subclass thereof (isotype), e.g., IgG1, IgG2, IgG3, IgG4, IgA1, and IgA2. The heavy chain constant domains that correspond to the different classes of immunoglobulins are called α, δ, ε, γ and μ, respectively. According to the present invention, an antibody of the class IgG, IgA or IgM or a fragment thereof is particularly suitable.

According to a preferred embodiment, an antibody of the invention is selected from class IgG, e.g. of subclass IgG1, IgG2, IgG3 of IgG4, of class IgM, of class IgA or an antigen-binding fragment thereof.

As used herein, the terms “binding” and “specific binding” refer to the binding of the inventive antibody or fragment thereof to an epitope of the Nectin-4 antigen. The measure of the binding strength of an antibody is referred to as affinity. Methods for determining such a binding and/or affinity using in vitro assays are known to the person skilled in the art. According to the present invention, detection with flow cytometry, immuno-histochemistry and/or fluorescence are described and in particular preferred herein.

The affinity of the binding of an antibody to an antigen is defined by the terms Ka (rate constant for the association of the antibody from the antibody/antigen complex), KD (dissociation constant), and K_{dis} (KD/ka), i.e. the term “Ka”, as used herein, refers to the association rate of a particular antibody-antigen interaction, whereas the term “Kd” refers to the dissociation rate of a particular antibody-antigen interaction. Such values for an antibody can also be determined using methods well established in the art and also described herein.

Antibodies according to the invention and antigen-binding fragments thereof preferably show a dissociation constant KD of at least 3.5, preferably at least 4.0, more preferably at least 4.2 and most preferably at least 4.5 (nM).

Of course, a specific binding to human Nectin-4 expressed by tumors is preferred. Thus, the antibodies provided herein show preferably specific binding to Nectin-4 and no essential or no cross reactivity to other proteins, in particular to proteins of the human Nectin-family such as Nectin-1.

The antibody or the antigen-binding fragment thereof may be produced in a suitable host cell comprising a nucleic acid molecule, e.g. a DNA molecule, encoding an antibody VH region, or an antibody VL region, or encoding a complete antibody or an antibody fragment, or a vector or vector system, i.e. a plurality of vectors, comprising said nucleic acid molecule(s), preferably in operative linkage with an expression control sequence, particularly with a heterologous expression control sequence. A method for providing the inventive antibodies is described herein. The host cell may be any known host cell for producing antibodies or antibody fragments, e.g. a prokaryotic cell such as an E. coli cell, a yeast cell, an insect cell or a mammalian cell, e.g. a CHO cell or a hybridoma cell.

Surprisingly, the inventive humanized antibodies, and in particular those characterized by VH regions comprising an amino acid sequence according to SEQ ID NO:2-5 and VL regions comprising an amino acid sequence according to SEQ ID: 6-9, may be provided in high yields compared to the corresponding parental chimeric 5A12.2 antibody (see Tables 2 and 3).

According to a further embodiment, inventive antibodies or antigen-binding fragments thereof comprise a labeling group and/or an effector group coupled to the antibody or antigen-binding fragment. The labeling group may be, for example, a dye, a paramagnetic, radioactive or fluorogenic group that is detectable upon imaging. A preferred effector group is a therapeutic group, in particular a cytotoxic agent, such as chemotherapeutically active agents, drugs, anti-inflammatory agents, radioactive isotopes, toxins such as topoisomerase poisons, enzymes and fragments thereof such as nucleolytic enzymes, growth inhibitory agents, antibiotics as wells as all suitable anticancer and antitumor agents known to the person skilled in the art. Especially preferred is the topoisomerase poison Camptothecin and derivatives and/or structural analogues thereof like Exatecan as well as derivatives thereof like Deruxtecan.

A further aspect of the invention relates to an antibody-drug conjugate (ADC), comprising a humanized antibody or antigen-binding fragment thereof as herein described and a drug conjugated to a reactive amino acid residue on the antibody or antigen-binding fragment, e.g., an amino acid residue having a side chain comprising an amino, hydroxy or thiol group, or a reactive group in the antibody glycan structure. Preferably, the drug is conjugated to a reactive thiol group in the side chain of a cysteine residue on the antibody or antigen-binding fragment thereof.

The drug of the antibody conjugate of the invention is preferably a topoisomerase I inhibitor, auristatin or maytansinoid.

A topoisomerase I inhibitor is a compound, which is capable of forming a ternary complex with topoisomerase I and DNA, thereby preventing DNA re-ligation and introducing DNA strand breaks in the cellular genome. The topoisomerase I inhibitor may be e.g., selected from camptothecin or analogs thereof, indenoisoquinolines and indolocarbazoles. In a particular embodiment, the topoisomerase-I-inhibitor is camptothecin or an analog thereof, i.e. a compound comprising the pentacyclic basic structure of camptothecin and modified substituents optionally resulting in the presence of a further ring. Specific examples are camptothecin, topotecan, irinotecan, SN-38, belotecan, exatecan including derivatives thereof such as deruxtecan, lurtotecan or atiratecan. In a particular preferred embodiment, the topoisomerase I inhibitor is exatecan. Exatecan is typically covalently conjugated to an antibody via its NH₂ group.

A particular preferred auristatin is monomethyl auristatin E (MMAE) which is a synthetic antineoplastic agent. MMAE inhibits cell division by blocking the polymerisation of tubulin.

Derivatives of maytansine are known as maytansinoids. Maytansinoids inhibit the assembly of microtubules by binding to tubulin at the rhizoxin binding site. Examples of maytansinoids are ansamitocin, Mertansine/emtansine (DM1) and Ravtansine/soravtansine (DM4).

In principle, the drug, e.g. exatecan, may be conjugated to any suitable position of the monoclonal antibody or antigen-binding fragment thereof, particularly to any position, which does not abolish the binding of the antibody to Nectin-4. For example, the topoisomerase-I-inhibitor, e.g. exatecan, may be conjugated to a reactive amino acid residue on the antibody, e.g. an amino acid residue having a side chain comprising an amino, hydroxy or thiol group, or a reactive group in the antibody glycan structure. In particular embodiments, drug, e.g. exatecan, is conjugated to a reactive thiol group in the side chain of an accessible cysteine residue on the antibody.

In certain embodiments, the antibody drug conjugate comprises has a molar drug-antibody/antibody fragment ratio (DAR) of greater than 1, i.e. more than one drug molecule is attached to an antibody/antibody fragment. Typically, the conjugate has a DAR of about 2:1 to about 16:1, particularly of about 4:1 to about 10:1 and more particularly of about 6:1 to about 8:1. The DAR may be calculated from a statistical distribution according to known methods.

In certain embodiments, the drug is conjugated to the antibody or antigen-binding fragment via a linker. In certain embodiments, the linker is a cleavable linker, i.e. a linker cleavable under physiological conditions, e.g. by physiological enzymes. Specific examples of cleavable linkers are peptide-based linkers, which may be subject to cleavage by a protease, or glycoside-based linkers, which may be subject to cleavage by a glycosidase.

In certain embodiments, the linker is a hydrophilic polysarcosine linker e.g. as described by Conilh et at. “Exatecan antibody drug conjugates based on a hydrophilic polysarcosine drug-linker platform”. (Pharmaceuticals 14 (2021), 247). Further preferred linkers include linkers comprising at least one ethylene glycol unit, e.g. 1, 2, 3, 4, 5, 6, 7, 8, 9, or 10 or more ethylene glycol units, e.g. the linker of the antibody-drug conjugate MEDI7247, an mcc-triazole spacer-PEG7-x-Lys-PABC glycol linker from Trodelvy®, Further preferred linkers include oligopeptide, particularly di- to decapeptide, e.g. tetrapeptide sequences such as glycine-glycine-phenylalanine-glycine from Enhertu®. Further preferred linkers include highly polar spacers such as an acyl group, carbamoyl group and/or sulfamide group added to at least at least one ethylene glycol unit, e.g. 1, 2, 3, 4, 5, 6, 7, 8, 9, or 10 or more ethylene glycol units, e.g. the linker technology called Hydraspace™.

The antibody drug conjugate of the present invention may be prepared by known methods.

The antibody or antigen-binding fragment thereof may be reacted with a linker-drug conjugate to obtain the antibody-drug conjugate. The linker-drug conjugate comprises a drug molecule, e.g. having attached thereto a suitable linker, wherein the linker comprises a reactive group capable of reacting with desired attachment positions on the antibody or antigen-binding fragment thereof. For attachment to cysteine residues, the linker-drug conjugate comprises a thiol-reactive group, e.g. a maleimide group.

A preferred embodiment of an antitumor agent relates to antitumor immune stimulating agents including but not restricted to toll-like receptor (TLR) agonists or stimulators of interferon genes (STING) pathways.

According to another preferred embodiment, an anti-inflammatory agent may be selected from group comprising steroids and corticosteroids such as glucocorticoids, e.g. cortisol and derivatives thereof, or mineralocorticoids such as aldosterone and derivatives thereof.

According to a further aspect of the invention, the antibody or antigen-binding fragment thereof is for use in medicine, particularly for therapeutic or diagnostic applications including in vitro and in vivo diagnostic applications.

The antibody or antigen-binding fragment thereof may be of use in a method of preventing and/or treating cancer and/or inflammatory disorders, wherein the cancer and/or inflammatory disorder is preferably associated with Nectin-4 overexpression.

The cancer to be prevented and/or treated may be any kind of cancer, wherein the term “cancer” is used herein to refer to proliferative diseases. The cancer to be prevented and/or treated according to the present invention is preferably selected from the group consisting of urothelial, endometrial, cervical, colorectal, liver, bladder, thyroid, breast, pancreatic, lung, ovarian head and neck and/or esophagus cancer.

Being specific for Nectin-4 expressed on tumors, the inventive antibodies and fragments thereof may be also used in diagnostics, for example being coupled to a labeling group as described above.

According to a further aspect, the antibody or antigen-binding fragment thereof as described herein may be used in a method of preventing and/or treating an inflammatory disorder, wherein the inflammatory disorder is preferably associated with Nectin-4 expression. Such treatment may be preferably combined with an anti-inflammatory agent known to the person skilled in the art. Preferred anti-inflammatory agents are described above.

Another aspect of the present invention is a combination of at least 2 different monoclonal antibodies or fragments as described herein.

Further, the present invention relates to a nucleic acid molecule, e.g. a DNA molecule, encoding an antibody VH region, or an antibody VL region, or encoding a complete antibody or an antibody fragment as indicated above, a vector or vector system, i.e. a plurality of vectors, comprising said nucleic acid molecule(s) as indicated above, preferably in operative linkage with an expression control sequence, particularly with a heterologous expression control sequence.

Furthermore, the invention relates to a cell comprising a nucleic acid molecule or a vector or vector system as described above. The term “vector,” as used herein, refers to a nucleic acid molecule capable of propagating another nucleic acid to which it is linked. According to a preferred embodiment, the vector is an expression vector. An “expression vector” is a vector are capable of directing the expression of nucleic acids to which they are operatively linked. Vectors, in particular expression vectors, for the recombinant production of antibodies are well known in the art.

The cell may be a known host cell for producing antibodies or antibody fragments, e.g. a prokaryotic cell such as an E. coli cell, a yeast cell, an insect cell or a mammalian cell, e.g. a CHO cell or a hybridoma cell.

Still a further aspect of the present invention relates to a pharmaceutical composition comprising an antibody or antigen-binding fragment thereof as described herein. Typically, the antibody or antibody-fragment is administered as a pharmaceutical composition comprising the active agent and a pharmaceutically acceptable carrier or excipient. Examples of suitable carriers and excipients for formulating antibodies or antibody fragments include saline and aqueous buffer solutions and are well known in the art.

Depending on the stage and the severity of the disorder to be treated, the pharmaceutical composition may be administered once or several times during the course of the disorder. For example, it may be administered daily, each second day, two times weekly or weekly for a suitable period of time.

In certain embodiments, the pharmaceutical composition is administered parenterally, e.g. by subcutaneous, intramuscular or intravenous injection or by infusion. In further embodiments, the pharmaceutical composition may be administered locally, e.g. orally, nasally or pulmonally, for example as an aerosol.

The invention is further illustrated by the following figures and examples, which are not intended to limit the scope of the invention.

### FIGURES

FIG. 1: Evaluation of the compatibility of antibodies 5A12.2_H1L0, HA22 (Enfortumab) and 15A7.5_H1L2

Yeasts expressing the Nectin-4 fragment 32-349 were incubated with 100 nM of unlabeled antibody 1 or PBS (as positive control). After 30 minutes incubation, 500 μM of antibody 2 was added for 30 minutes. Yeasts were then incubated with fluorescent reporters to detect by FACS Nectin-4 expression and antibody 2. Results are expressed as percentage of fluorescence to the respective positive control

FIG. 2: Libraries sorting by flow cytometry

Two libraries covering the Nectin-4 sequence of interest were generated (left and right panels). Yeasts expressing Nectin-4 mono-mutants were incubated either with 100 μM of biotinylated 5A12.2_H1L0 and 100 μM of 15A7.5_H1L2 labelled with Dy650 (upper panels), or 100 μM of biotinylated HA22 and 100 μM of 15A7.5_H1L2 labelled with Dy650 (lower panels). Streptavidin PE was used to reveal biotinylated antibodies. After PBS washing, cells were analyzed and sorted on BD FACSAria III as indicated on the graphs. Single-stained yeasts by an antibody express Nectin-4 mono mutants that disrupt the epitope for the binding of the other antibody.

FIG. 3: Positions showing a significant impact on the 5A12.2_H1L0 antibody when mutated.

For any amino acid of the domain of interest, the higher the number of forbidden mutations, the more important the residue for the binding of 5A12.2_H1L0 antibody.

FIG. 4: Positions showing a significant impact on the HA22 (Enfortumab) antibody when mutated.

For any amino acid of the domain of interest, the higher the number of forbidden mutations, the more important the residue for the binding of HA22 antibody.

FIG. 5: Structure reference of Nectin-4 (4FRW, pdb)

Shown are pymol files. For each antibody, important amino acids of the epitope are indicated. Most important residues are underlined.

FIG. 6: in vitro cytotoxic activity to SUM190PT and T47D tumor cells

HA22 (Enfortumab, circles), Ch-5A12.2 (triangles) and Isotypic control (open squares) mAbs were coupled to α-amanitin to generate antibody drug conjugates which cytotoxic activity of a dose range (left: 1 pg/mL-15 μg/mL, right: 192 pg/mL-15 μg/mL) was evaluated on SUM190PT (left) and T47D (right) Nectin-4-expressing cell lines. Viability (AlamarBlue assay) after a 5-days incubation period is reported. EC50 values were determined by GraphPad Prism 9 software using non-linear curve fitting (4 parameters).

FIG. 7: Apparent affinity of humanized variants to tumor cells. Detection by flow cytometry.

T47D human tumor cells expressing Nectin-4 were numbered and incubated with a dose range (169 μg/mL-30 μg/mL) of Ch-5A12.2 or indicated humanized variants. Numbers associated with H and L refer to the number of back mutations introduced. Cells were then stained with phycoerythrin conjugate goat anti human Fc antibody and analyzed by flow cytometry. Mean fluorescence intensities are reported. GraphPad Prism 9 software was used with non-linear curve fitting (4 parameters).

FIG. 8: in vitro cytotoxic activity to HCT-116 transfectant expressing Nectin-4

Hu-5A12.2_H1L0 (triangles), Hu-5A12.2_H1L1 (open diamonds), Hu-15A7.5_H1L2 (circles) and isotypic control (ICT, open squares) mAbs were coupled to exatecan to generate antibody drug conjugates which cytotoxic activity of a dose range (left: 1 pM-18.94 nM) was evaluated on HCT-116 Nectin-4-expressing cell lines. Viability (MTT assay) after a 7-days incubation period is reported. EC50 values were determined by GraphPad Prism 9 software using non-linear curve fitting (4 parameters).

FIG. 9: Treatment of SUM190PT grafted NSG mice with Hu-15A7.5_H1L2-Exatecan or Hu-5A12.2_H1L0-Eatecan ADC induce tumor regression period.

NSG mice (n=5/group) were orthotopically xenografted bilaterally with the SUM190PT cells embedded in Matrigel. Three different ADC were tested: Isotypic Control, ICT-Exatecan (black circles), Hu-5A12.2_H1L0-Exatecan (open diamonds) and Hu-15A7.5_H1L2-Exatecan (black diamonds). Treatment of mice (single intra-venous injection 10 mg/kg) started when tumors reached approximately 70 mm³. Tumor sizes (n=10/group) were monitored with a caliper twice a week thereafter and sizes were reported with the following formula (LxlxhxPi/6).

FIG. 10: Amino acid sequence of the parental 5A12.2 monoclonal antibody Shown are the heavy and light chain amino acid sequences of the parental 5A12.2 monoclonal anti-Nectin-4 antibody. CDRs are identified according to IMGT and Kabat nomenclature

(IMGT nomenclature, Kabat nomenclature)

FIG. 11: Amino acid sequence of humanized variants of the 5A12.2 monoclonal antibody

Show are the heavy and light amino acid sequences of the humanized variants of the 5A12.2 monoclonal anti-Nectin-4 antibody. CDRs are identified according to IMGT and Kabat nomenclature. Back mutations are also identified. (IMGT Nomenclature/Kabat nomenclature/anchor IMGT and/or Kabat/^{Back mutation})

FIG. 12: Summarized sequences of the inventive humanized antibodies

### EXAMPLES

### Principle of Epitope Mapping Using DMS (Deep Mutational Scanning)

DMS is a mutagenesis method that aims to perform all possible mono-substitutions on all selected residues within a given protein sequence. The DMS library is obtained in the form of DNA coding for the protein under study. In this library, each DNA strand contains a codon that is mutated with respect to the parental sequence.

This DMS DNA library is integrated into an expression plasmid specifically designed to express recombinant proteins on the yeast surface. Yeasts are then transformed and induced to allow the expression of the mono-mutated proteins on their surface. This new library (called display library) is screened by flow cytometry using fluorescent reporters to reveal the expression of the protein (anti-tag fluorescent antibody) as well as the binding of the protein to its partner (fluorescent partner).

For epitope mapping, the ideal case is to have two antibodies with compatible epitopes that can bind together on the same antigen. In this way, each of the two antibodies acts as a conformational control of the mutated antigen for the other antibody. Indeed, mono-substitutions made on the antigen can have 4 types of effects: (i) loss of affinity for the first antibody, while retaining binding for the second: this is a mutation made within the epitope of the first antibody; (ii) loss of affinity for the second antibody, while retaining binding for the first: this is a mutation in the epitope of the second antibody; (iii) loss of affinity for both antibodies: this is a so-called “destructuring” mutation which affects the conformation of the antigen and thus prevents the binding of both antibodies; (iv) no effect, the mutation is not present in the epitope of one of the two antibodies and does not cause a significant change in the conformation of the antigen. Following flow cytometry analysis, the yeast population that has lost affinity for the antibody of interest while retaining binding for the second antibody is sorted. The plasmids contained in this yeast population are extracted and sequenced by high-throughput sequencing. Analysis of the sequencing data allows the identification of mutations that have affected the binding of the antibody to its target. Thus, this analysis allows to identify the important positions on the antigen for the binding of the antibody of interest: i.e. its epitope.

### Construction of the Expression Plasmid of the Antigen

The gene corresponding to the antigen sequence Nectin-4 (32-349) was synthesized and cloned into a plasmid allowing expression on the surface of galactose-inducible yeast. In this construct, the expressed antigen carries a C-terminal HA tag. Expression plasmids are then transformed into the yeast strain S. cerevisiae EBY100. Two DMS libraries were generated by PCR, concerning amino acid positions 32 to 91 for Library 1 and amino acid positions 92 to 151 for Library 2. The libraries correspond to IgV domain of Nectin-4, which is known to be the target of the tested antibodies. Each mutated position carries a degenerated NNS or NNK codon encoding 20 amino acids/32 codons. Each library contained approximately 1,200 single amino acid mutants and 2,000 DNA codon mutants. The two libraries were transformed into YSD. Unsorted yeasts from the two generated libraries were sequenced to verify the efficiency of mutagenesis. One hundred percent of the expected single mutants were sequenced for library 1 and 99.9% for library 2.

### Epitope Compatibility

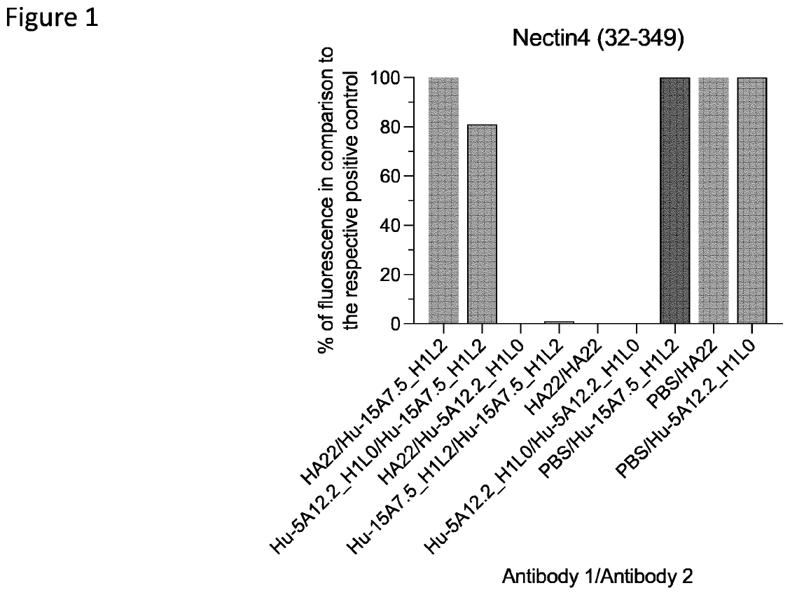

To check if the tested antibodies were compatible for epitope mapping on Nectin-4 IgV domain, i.e. that the antibodies can bind together on Nectin-4 IgV domain, antibodies were first biotinylated (EZ-Link™ Sulfo-NHS-LC-Biotin). Yeasts were induced to express Nectin-4 32-349 construct and 105 yeasts were washed twice with PBS, BSA 0.1%. Yeast cells were then incubated 30 minutes with 100 nM of unlabeled antibody 1 or left as is for positive control. After 30 minutes, 500 μM of antibody 2 was added for another 30 minutes incubation. Yeasts were then incubated on ice for 15 minutes with respective fluorescent reporters, anti-HA APC for Nectin-4 expression detection and Streptavidin-PE for antibody 2 detection. After washing in PBS-BSA, resuspended yeasts were analyzed by flow cytometry. FIG. 1 shows that 5A12.2_H1L0 and HA22 cannot bind simultaneously to Nectin-4 IgV domain. In contrast, 15A7.5_H1L2 mAb can bind Nectin-4 IgV domain simultaneously with either 5A12.2_H1L0 or HA22 (Enfortumab).

### Induction of Antigen Expression and FACS-Sorting of Libraries

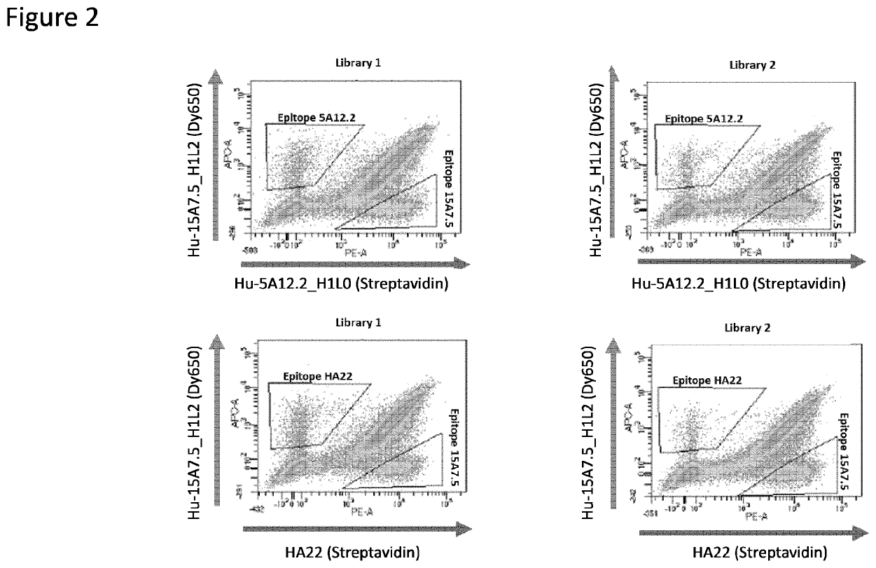

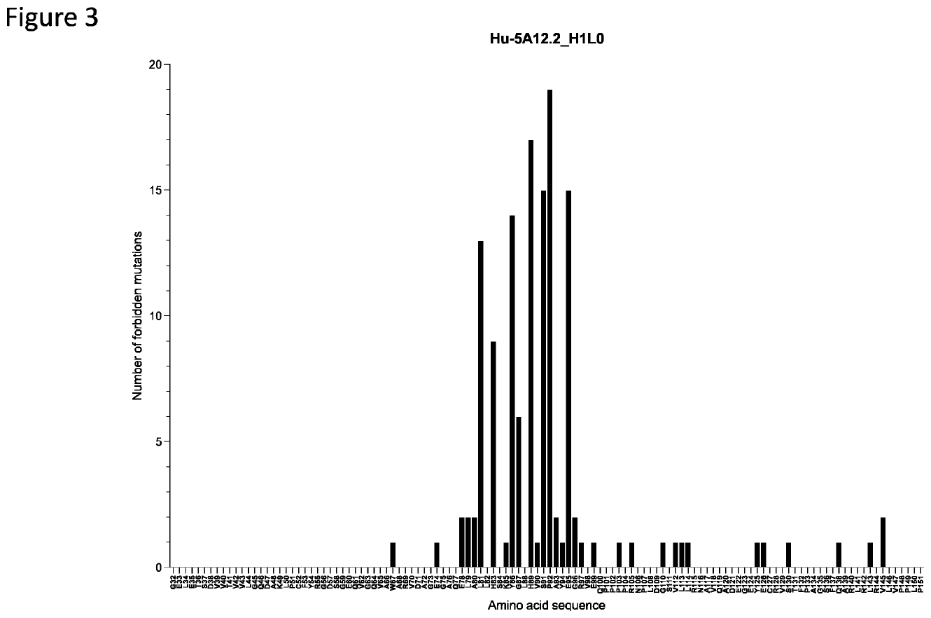

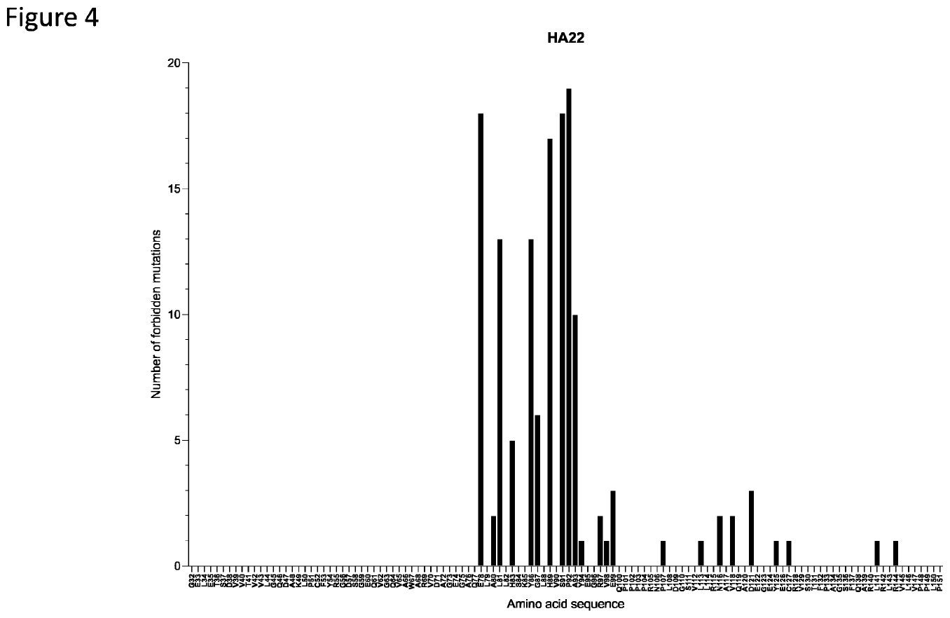

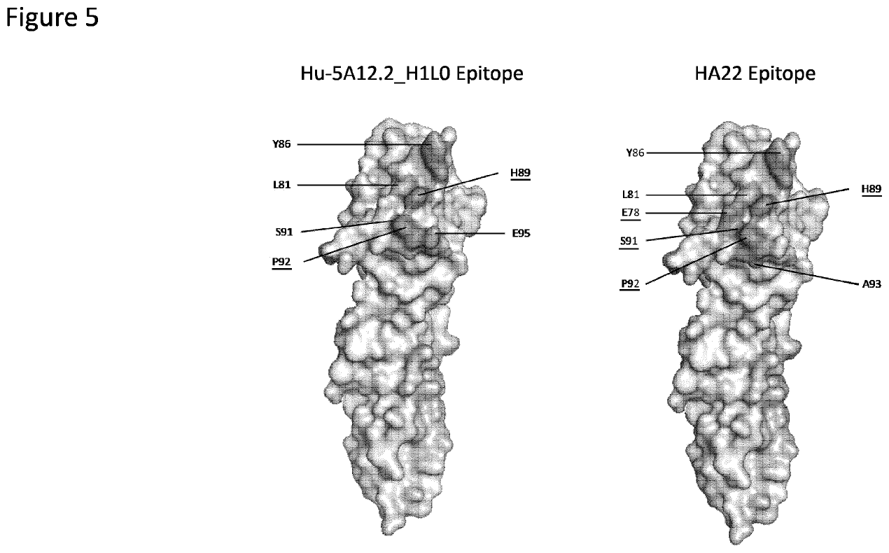

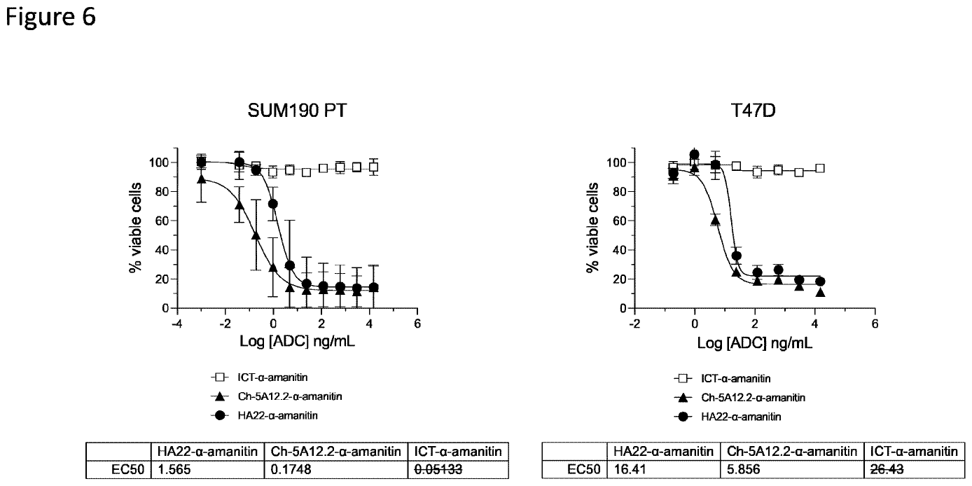

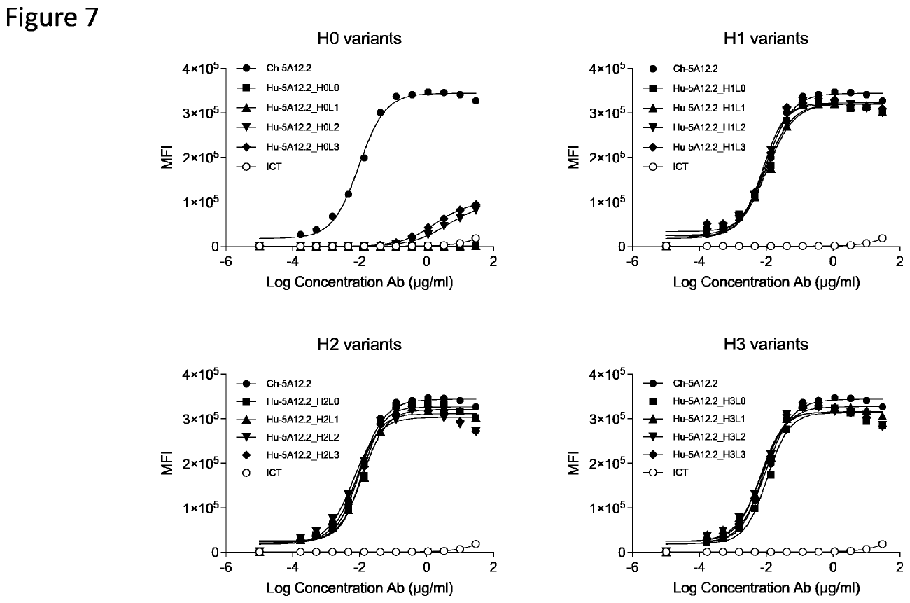

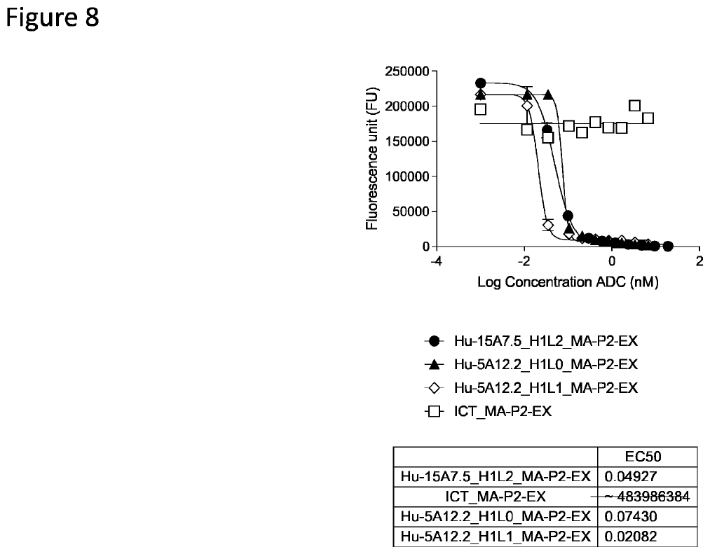

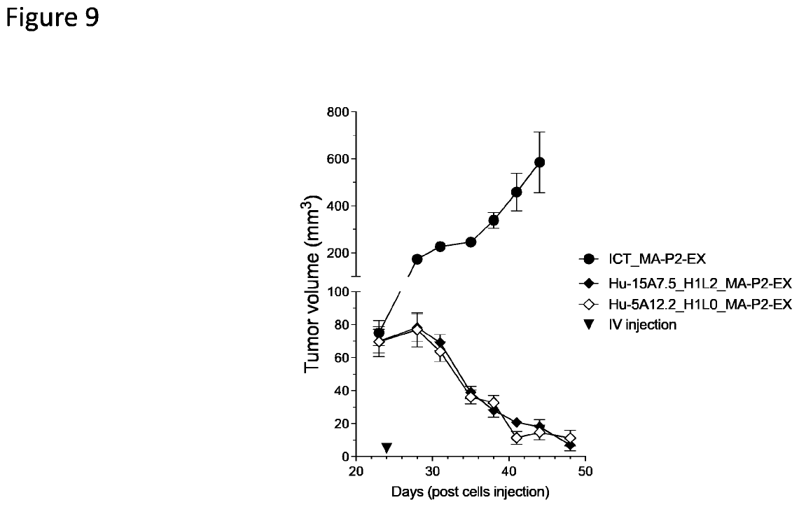

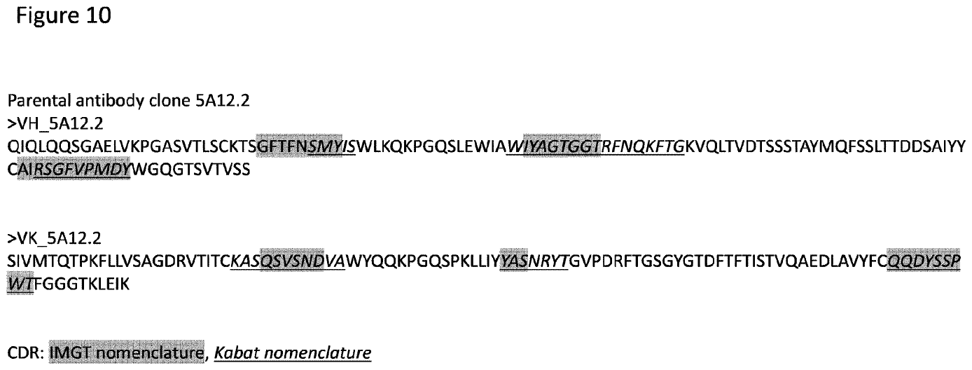

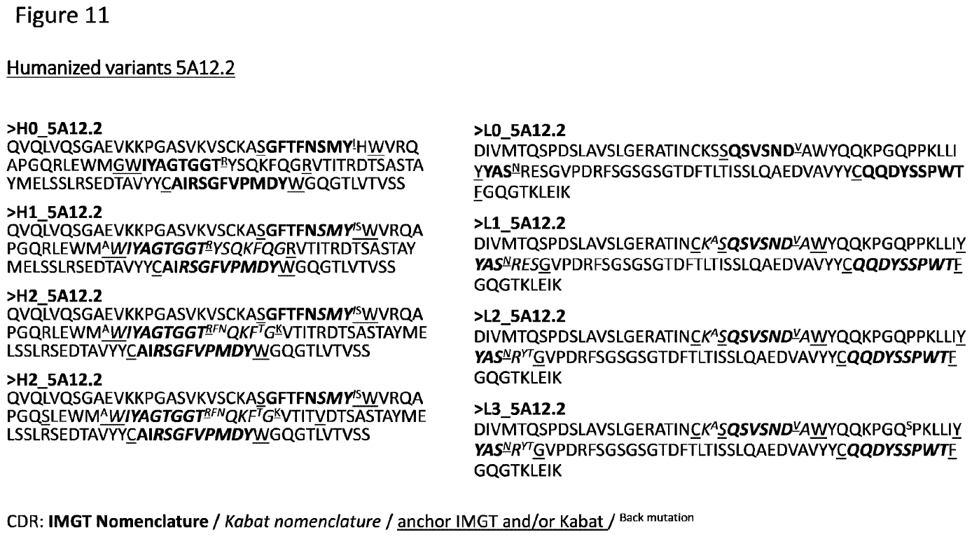

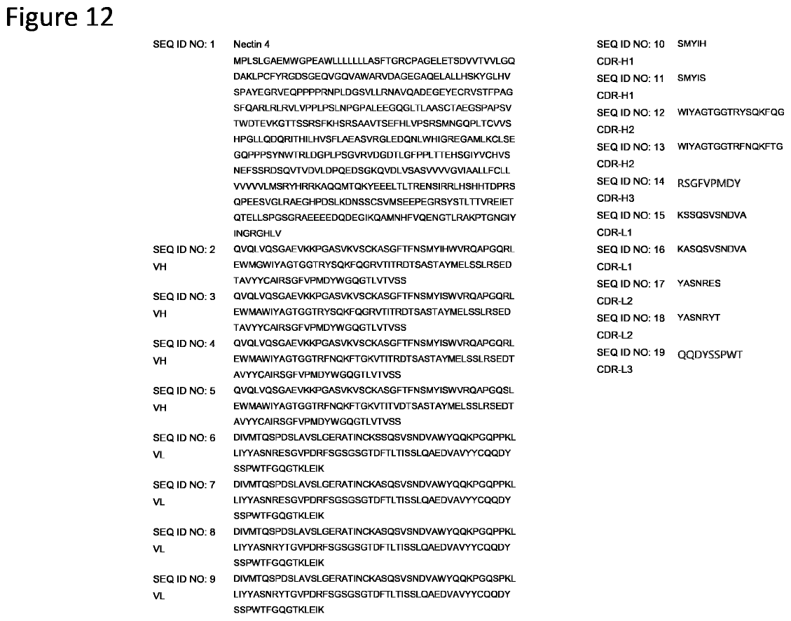

Yeasts expressing Nectin4 mono-mutants were induced for protein expression. Induction of transformed yeast was done in SG-CAA induction medium [6.7 g/L yeast nitrogen base without casamino acids, 20 g/L glucose, 5 g/L casamino acids, 100 mM sodium phosphate, pH 6.0]. For cytometric analysis/sorting, between 10⁶ and 10⁸ induced cells were washed with 1 mL of PBSF (PBS, 0.1% BSA). The cells were then resuspended in an appropriate volume of solution containing 100 μM of the biotinylated HA22 antibody and Dye650-labeled 15A7.5_H1L2. After 1 h to 3 h of incubation at 20° C. with agitation, the cells were washed with 1 mL of ice-cold PBSF and then incubated with the streptavidin on ice for 15 min. The cells were then analyzed/sorted on a BD FACSAria™ III cytometer using the BD FACSdiva™ Diva software. As shown in FIG. 2, the single-stained yeasts in each experiment were sorted and plasmids from each sorted yeast population were extracted and prepared for sequencing. A two-step PCR was performed: a first step to amplify the region of interest and a second step to add the Illumina adapters needed for sequencing. Sequencing was performed on an Illumina iSeq100 instrument (2×150 bp, 300 cycles) with at least 150,000 reads per population. The data were then processed through an analysis pipeline using dedicated proprietary scripts. Poor quality sequences (Q<30) were removed, then mono-mutants were detected and counted. For each mono-mutation, the frequency measured in the unsorted population was compared to the frequency of the mutant in the sorted population. An enrichment value for the mutant was then calculated according to the following formula:

$$Enrichment mutant = \frac{Frequency mutant , sorted population}{Frequency mutant , unsorted population}$$

A high enrichment value indicates that the mutation under consideration caused a loss of recognition of the antibody for its antigen, while maintaining recognition of the other antibody (conformational control).

### Data Interpretation

The result of the processing of the NGS sequencing data is represented as a bar graph that aggregates the enrichment values for all single mutants present in the DMS libraries (FIGS. 3, 4). Between 16 and 19 forbidden mutations indicate high impact and positions classified in this category are the most likely to be indirect interaction with the considered IgG. Between 10 and 15 forbidden mutations indicate medium impact positions, and between 5 and 9 forbidden mutations indicate low impact positions. FIG. 5 shows an analysis of the structure of Nectin-4 (4FRW, pdb) to highlight the important and most important epitope residues for each of the antibody tested. Table 1 lists these residues.

TABLE 1
Important and most important residues of the Nectin-4
epitopes of monoclonal antibodies HA22 and 5A12.2_H1L0

| | | Most important epitope |
|---|---|---|
| Antibody | Epitope residues | residues |
| HA22 | E78, L81, H83, Y86, G87, | E78, H89, S91 and P92 |
| | H89, S91, P92 and A93 | |
| 5A12.2_H1L0 | L81, H83, Y86, G87, H89, | H89 and P92 |
| | S91, P92 and E95 | |

### Humanization of Chimeric Antibodies

The methodology used is the “CDR grafting”: the 3 mouse CDR (regions determining the specificity of the antibody) of each antibody chain (heavy, VH or light, VL) are grafted on the closest human germline backbone. This methodology takes also in consideration the back-mutations of essential amino-acids (anchors of the CDR, Vernier's residues, interface VH/VL, etc. . . . ) in the FrameWork regions (FR).

### Chimeric and Humanized Antibodies Generation, Production, Purification and Control

Light chain expression vector is coding for a V kappa chain. Depending on the payload used, 2 different heavy chain expression vectors were used, one coding for a Fc fragment with D265C (ThiomAb) L234A and L235A mutations, one coding for a Fc fragment with P331S, L234F and L235E mutations. Both Fc fragments are “Fc-silent”. The sequences of anti-Nectin-4 Enfortumab (HA22) were also cloned in the same vectors.

Light and heavy chain vectors were transfected in HEK293 seeded with a 1.2/1 ratio. After 6 days of production, culture supernatants were clarified and mAbs were purified using MabSelect PrismA resin (GE Healthcare) according to the manufacturer's instructions. 0.5M Glycine, 3M NaCl, pH8.9 was used as binding buffer, and 0.1 M Citrate pH 3 was used for elution. Instant neutralization was done with 10% (V/V) 1M Tris-HCl pH 9. Monoclonal antibodies were then dialyzed against PBS 1× pH 7.4 (Mini dialysis devices, 2 mL-10k, Thermo Scientific) followed by filtration on 0.22 UM filter (Milelex GV hydro-philic PVDF, Millipore). Concentration was determined with a Nanodrop 2000 Spectrophotometer (Thermo Scientific) considering the specific extinction coefficient (E1% 280 nm) of each monoclonal antibody. Purity was determined by UPLC-SEC using an Acquity UPLC-HClass Bio (Waters) using a Protein-BEH 200A column equilibrated in 0.2 M NaPO4, 0.3 M NaCl pH 6.9 supplemented with 10% isopropanol. The mass of the antibodies was determined in a Xevo G2-S Q-Tof mass spectrophotometer (Waters) using a reversed-phase column (PLRP-S 4000A, Agilent technologies). All samples were analyzed after deglycosylation with PNGase F glycosidase (New England Biolabs) at 37° C., according to the manufacturer's instructions. Fragmentation and/or aggregation of the final material was evaluated by SDS-PAGE. Endotoxin load was determined using a chromogenic LAL-kinetic assay (Charles River Endosafe).

Table 2 reports production yields at small scale (30 mL) of the parental chimeric 5A12.2 and all the humanized variants.

Table 3 reports production yields at medium scale (0.5-1.4 L) of the parental chimeric 5A12.2 and humanized variants 5A12.2_H1L0 and 5A12.2_H1L1.

TABLE 2
Small scale production yield of humanized variant
versus that of parental chimeric 5A12.2 antibody.

| | Amount produced | Volume | Yield |
|---|---|---|---|
| Antibody | (mg) | (mL) | (mg/mL) |
| CHt_5A12.2 | 2.21 | 30 | 0.074 |
| HUt_5A12.2_H0L0 | 5.94 | 30 | 0.198 |
| HUt_5A12.2_H0L1 | 6.1 | 30 | 0.203 |
| HUt_5A12.2_H0L2 | 5.77 | 30 | 0.192 |
| HUt_5A12.2_H0L3 | 6.07 | 30 | 0.202 |
| HUt_5A12.2_H1L0 | 5.41 | 30 | 0.180 |
| HUt_5A12.2_H1L1 | 5.15 | 30 | 0.172 |
| HUt_5A12.2_H1L2 | 6.08 | 30 | 0.203 |
| HUt_5A12.2_H1L3 | 5.60 | 30 | 0.187 |
| HUt_5A12.2_H2L0 | 4.44 | 30 | 0.148 |
| HUt_5A12.2_H2L1 | 4.73 | 30 | 0.158 |
| HUt_5A12.2_H2L2 | 5.73 | 30 | 0.191 |
| HUt_5A12.2_H2L3 | 3.54 | 30 | 0.118 |
| HUt_5A12.2_H3L0 | 5.15 | 30 | 0.172 |
| HUt_5A12.2_H3L1 | 5.95 | 30 | 0.198 |
| HUt_5A12.2_H3L2 | 5.74 | 30 | 0.191 |
| HUt_5A12.2_H3L3 | 6.07 | 30 | 0.202 |

CHt: chimeric antibody,
HUt: humanized antibody

TABLE 3
Medium scale production yield of humanized variant
versus that of parental chimeric 5A12.2 antibody.

| | Amount produced | Volume | Yield |
|---|---|---|---|
| Antibody | (mg) | (mL) | (mg/mL) |
| CHt_5A12.2 | 129.5 | 1400 | 0.093 |
| HUt_5A12.2_H1L0 | 76.5 | 500 | 0.153 |
| HUt_5A12.2_H1L1 | 83.1 | 500 | 0.166 |

CHt: chimeric antibody,
HUt: humanized antibody

### Antibody Conjugation

A cysteine reactive linker-amanitin compound from Heidelberg Pharma with a cleavable linker (valine alanine) to engineered cysteine residues of selected anti-Nectin-4 ThiomAb (D265C, L234A, L235A) antibodies using maleimide chemistry. Briefly, ThiomAb antibodies in PBS 1× pH 7.4 were reduced with TCEP and interchains disulfides were re-oxidized by dehydroascorbic acid. Subsequently, the engineered cysteines were used for conjugation with cysteine reactive linker amanitin compound. The conjugates were purified by dialysis. The drug-antibody ratio (DAR) according to LC-MS analysis was comprised between 1.44 and 1.79 toxins per conjugated mAb. As determined by SEC-HPLC, less than 2% material was aggregated.

The cysteine reactive linker-exatecan compound Maleimide-Propio-PEG2-PSAR10-glucuronide-exatecan (MabLink) was conjugated to cysteine residues of selected anti-Nectin-4 mAbs (P331S, L234F and L235E). In brief, mAbs in PBS 1×, 1 mM EDTA were reduced with 14 molar equivalent of TCEP for 2 hours at 37° C., after which the buffer was exchanged (Amicon ultra 30 kDa) to 100 mM KPO₄, 1 mM EDTA PH 7.4. Twelve molar equivalents of the cysteine reactive linker-exatecan compound were used for conjugation with reactive cysteines for 35 min at room temperature. The final exchange buffer was performed in 20 mM His pH 6.0 before filtration 0.22 UM filter. The drug-antibody ratio (DAR) according to LC-MS analysis was comprised between 7.77 and 7.82 toxins per conjugated mAb. As determined by SEC-HPLC, less than 8% material was aggregated.

### Cell Lines

Human triple negative breast cancer cell line SUM190PT (BioIVT, Westbury, NY) was cultured in Ham's F12 medium with 5% fetal bovine serum, 1% non-essential amino acids, 1% Hepes, 1% insulin, 1 μg/mL hydrocortisone, 6.8 ng/ml Triiodo L-tyrosine, 100 IU/mL penicillin, 100 μg/mL streptomycin and 2 mM glutamine. Human breast carcinoma T47D cell line was cultured in RPMI supplemented with 10% fetal bovine serum, 100 IU/mL penicillin, 100 μg/mL streptomycin. The Human colorectal carcinoma Nectin-4 transfectant (HCT116-N4) was cultured in RPMI supplemented with 10% fetal bovine serum, 2 mM L-Glutamine, 1×MEM Non-essential amino acids and 20 μg/mL Hygromycin B.

### In Vitro Cytotoxicity Assay

To analyze the in vitro cytotoxic activity of α-amanitin ADC and Exatecan ADC, cell viability was assessed using the AlamarBlue staining protocol as recommended by the manufacturer (Biosource, CA, USA). The test incorporates a fluorescent oxidation-reduction indicator. Fluorescence intensity is proportional to cellular metabolic reduction. Experiments were done by incubating 3,000 cells/well (SUM190PT, T47D or HCT116-Nectin-4) in triplicate with serial dilutions of ADC at Day 0 in 96-wells plates. AlamarBlue was measured at Day 5 by incubating 1/10 volume of AlamarBlue solution for 2 h at 37° C. and read at 595 nm (FLUOstar Optima, BMG Labtech). FIG. 6 shows that the parental 5A12.2 anti-Nectin-4 monoclonal antibody has a lower EC50 than HA22. FIG. 8 shows that humanized variants H1L0 and H1L1 of monoclonal antibody 5A12.2 display similar EC50 in comparison to another humanized anti-Nectin-4 antibody.

### Flow Cytometry

T47D cells (50,000) naturally expressing Nectin-4 were incubated with dose range of the indicated antibodies. After washing, cells were then stained with phycoerythrine-conjugated goat anti human antibody (5 μg/mL) Jackson Immuno Research). After fixation, cells were stained with a viability dye (e780, Invitrogen) before flow cytometry acquisition. FIG. 7 shows that all 5A12.2 humanized variants except H0 variants have retained similar apparent affinity in comparison to the parental 5A12.2 monoclonal antibody.

### Mouse Experiments

NOD/SCID (nonobese diabetic/severe combined immunodeficient)/gc null mice (NSG) were obtained from Charles River Laboratory (Margate, UK). Six to seven-weeks old females (n=5/group) were orthotopically xenografted bilaterally with the SUM190PT (0.5×10⁶) cells embedded in Matrigel. Treatment with ADC was performed as mentioned in the respective experiments. Tumor sizes (n=10/group) were monitored with a caliper twice a week thereafter and sizes were reported with the following formula (LxlxhxPi/6). FIG. 9 shows that, in vivo, a single injection of humanized variant 5A12.2_H1L0 monoclonal antibody coupled to Exatecan can induce regression of triple negative breast cancer cell line SUM190PT.

### Determination of Antibody Affinities on Octet Platform

Affinities of the various monoclonal antibodies were determined on the Octet Red96 platform which is a system based on bio-layer interferometry (BLI) technology. Two types of analytes were used in these studies: (i) a his-tagged recombinant extra cellular domain of the Nectin-4 protein (R&D systems/Biotechne); (ii) a homodimer recombinant Fc protein, corresponding to the IgV domain of human Nectin-4 fused to human Fc domain. An AHC sensor (anti human Fc) was used for the recombinant his-tagged Nectin-4 EC analyte and a FAB2G sensor was used for the Fc fusion recombinant IgV domain of Nectin-4. The ligand of interest diluted in 1× kinetic buffer is loaded on the selected sensor. The analyte remains in solution at working concentrations in 1× kinetic buffer ranging from 600 nM to 1.56 nM. Each run is performed at +26° C. under 1,000 rpm shaking and is made of 5 steps; loading step, equilibration step, association step, dissociation step and regeneration. For the analysis of generated data, the standard 1:1 Langmuir model was chosen for the AHC sensor and the 1:2 bivalent analyte model was chosen for the FAB2G sensor. Both analysis models are run by the Octet software. Table 2 indicates that the affinity for His-tagged recombinant Nectin-4 is similar for 5A12.2 and HA22 monoclonal antibodies. In contrast, the affinity for the Fc fusion IgV Nectin-4 domain is markedly different between 5A12.2 and HA22 suggesting that 5A12.2 has a better avidity for its dimeric epitope on Nectin-4 than HA22.

TABLE 4
K_{D} on 5A12.2 and HA22 monoclonal antibodies

| Antibody | K_{D} for His-Nectin-4 | K_{D} for Fc-Nectin-4 |
|---|---|---|
| Ch-5A12.2 | 4.2 nM | 5.3 nM |
| HA22 | 5.2 nM | 12.9 nM |

Table 5 reports the affinity determination for the various humanization variants of monoclonal antibody 5A12.2. Only the recombinant his-tagged Nectin-4 EC analyte was used here. The affinity of the various humanized variant is similar to that the parental antibody.

TABLE 5
Affinity determination of various humanized variants
of 5A12.2 in comparison to the parental antibody

| Antibody | K_{D} (nM) | Ka (1/Ms) | Kdis (1/s) | R² |
|---|---|---|---|---|
| Ch-5A12.2 | 3.4 | 2.75E+05 | 9.28E−04 | 0.99 |
| Hu-5A12.2_H1L0 | 4.6 | 2.66E+05 | 1.23E−03 | 0.98 |
| Hu-5A12.2_H1L1 | 4.7 | 2.67E+05 | 1.24E−03 | 0.98 |
| Hu-5A12.2_H2L0 | 4.4 | 3.6E+05 | 1.6E−03 | 0.96 |
| Hu-5A12.2_H2L1 | 4.2 | 3.89E+05 | 1.64E−03 | 0.95 |
| Hu-5A12.2_H3L0 | 4.3 | 3.4E+05 | 1.45E−03 | 0.96 |
| Hu-5A12.2_H3L1 | 4.2 | 3.49E+05 | 1.46E−03 | 0.96 |

1. A humanized monoclonal antibody or antigen-binding fragment thereof, which binds to a discontinuous epitope on Nectin-4 consisting of amino acids L81, H83, Y86, G87, H89, S91, P92 and E95 of SEQ ID NO: 1 as determined using Deep Mutational Scanning (DMS).

2. The humanized antibody according to claim 1, comprising
(a) a variable heavy chain (VH) region comprising an amino acid sequence according to any one of SEQ ID NOs: 2-5, or an amino acid sequence having an identity thereto of at least 85%, at least 90%, at least 95% or at least 99%,
and including complementarity-determining regions (CDRs) CDR-H1 according to SEQ ID NO: 10 or 11, CDR-H2 according to SEQ ID NO: 12 or 13 and CDR-H3 according to SEQ ID NO: 14,
(b) a variable light chain (VL) region comprising an amino acid sequence according to any one of SEQ ID NOs: 6-9, or an amino acid sequence having an identity thereto of at least 85%, at least 90%, at least 95% or at least 99%,
and including CDR-L1 according to SEQ ID NO: 15 or 16, CDR-L2 according to SEQ ID NO: 17 or 18 and CDR-L3 according to SEQ ID NO: 19.

3. The humanized antibody according to claim 2, comprising
(i) a VH region comprising an amino acid sequence according to SEQ ID NO: 2 or an amino sequence having an identity thereof of at least 85%, at least 90%, at least 95% or at least 99%, and
a VL region comprising an amino acid sequence according to SEQ ID NO: 6 or an amino sequence having an identity thereof of at least 85%, at least 90%, at least 95% or at least 99%,
(ii) a VH region comprising an amino acid sequence according to SEQ ID NO: 3, or an amino sequence having an identity thereof of at least 85%, at least 90%, at least 95% or at least 99%, and
a VL region comprising an amino acid sequence according to SEQ ID NO: 7 or an amino sequence having an identity thereof of at least 85%, at least 90%, at least 95% or at least 99%,
(iii) a VH region comprising an amino acid sequence according to SEQ ID NO:4 or an amino sequence having an identity thereof of at least 85%, at least 90%, at least 95% or at least 99%, and
a VL region comprising an amino acid sequence according to SEQ ID NO: 8, or an amino sequence having an identity thereof of at least 85%, at least 90%, at least 95% or at least 99%,
(iv) a VH region comprising an amino acid sequence according to SEQ ID NO: 5, or an amino sequence having an identity thereof of at least 85%, at least 90%, at least 95% or at least 99%, and
a VL region comprising an amino acid sequence according to SEQ ID NO: 9, or an amino sequence having an identity thereof of at least 85%, at least 90%, at least 95% or at least 99%.

4. The humanized antibody according to claim 2, comprising
(a) a VH region comprising an amino acid sequence according to SEQ ID NO:2-5, and
(b) a VL region comprising an amino acid sequence according to SEQ ID NO: 6-9.

5. The humanized antibody or antigen-binding fragment of claim 1, which is a multispecific antibody or an antigen-binding fragment thereof.

6. The humanized antibody or antigen-binding fragment of claim 1, which is an antibody of class IgG, or an antigen-binding fragment thereof, or which is a single-chain antibody, or an antibody Fv fragment.

7. The humanized antibody or antigen-binding fragment of claim 1, comprising a labeling group being coupled to the antibody or antigen-binding fragment.

8. The humanized antibody or antigen-binding fragment of claim 7, wherein the labeling group is a dye, a paramagnetic, radioactive or fluorogenic group that is detectable upon imaging.

9. (canceled)

10. (canceled)

11. (canceled)

12. (canceled)

13. (canceled)

14. (canceled)

15. (canceled)

16. (canceled)

17. A nucleic acid coding for the antibody according to claim 1.

18. A vector comprising the nucleic acid according to claim 17.

19. A host cell comprising the vector of claim 18.

20. A pharmaceutical composition comprising a humanized antibody or antigen-binding fragment thereof according to claim 1, and optionally one or more pharmaceutical excipients.

21. A method of treating a disease or disorder associated with Nectin-4 expression in a patient in need thereof, the method comprising administering to the patient the humanized antibody of claim 1 and optionally one or more pharmaceutical excipients.

22. The method of claim 21, wherein the disease or disorder is wherein the cancer is urothelial, endometrial, cervical, colorectal, liver, thyroid, breast, pancreatic, lung, ovarian, head and neck, or esophagus cancer.
